SEQUENCE LISTING (8 sequences)

SEQ ID NO: 1 - protein, 28 aa, Artificial
  Polypeptide 181-208 from gp41 HIV1
LVGLRIVFAVLSIVNRVRQGYSPLSFQT

SEQ ID NO: 2 - protein, 345 aa, Human immunodeficiency virus type 1
  MISC_FEATURE (1)..(345): Artificial
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 3 - protein, 114 aa, Artificial
  VL Variable region sequence
ELTQPSSLSASPGASASLTCTLRSGINVGTYRIYWYQQKPGSPPQFLLRYKSDSDKEQGSGVPRRFSGSKDASANAGILLISGLQSEDEADYYCMIWHSGAWVFGGGTKLTVLS

SEQ ID NO: 4 - protein, 124 aa, Artificial
  VH Variable region sequence
LESGGGMVQPGRSLRLSCAVSGFTFDDYAMHWIRQVPGKGLEWVAGISWNSVKVDYADAVRGRFTISRDNAKNSLHLEMSNLRRDDTAFYFCAKDGGPGMTIGGYVVTGRFDPWGQGTLVHVSS

SEQ ID NO: 5 - protein, 28 aa, Artificial
  gp41 Polypeptide
LVGLRIVFXVLSXVNRVRQGYSPLSFQT

SEQ ID NO: 6 - protein, 28 aa, Artificial
  gp41 polypeptide
LXGLRIXFXVXSXXXRVRQGYSPXSXQX

SEQ ID NO: 7 - protein, 29 aa, Artificial
  gp41 polypeptide
LXGLRIVFXXLSXXXXRVRQGYSPXSXQT

SEQ ID NO: 8 - protein, 9 aa, Artificial
  gp41 polypeptide
XXXXXXXXX

(19) United States
(12) Patent Application Publication
(10) Pub. No.: US 2010/0111990 A1
(43) Pub. Date: May 6, 2010

(54) Polypeptide Recognized by Anti HIV-1 GP41 Antibodies Isolated from Highly HIV-1 Exposed, Non Infected Women

(75) Inventors: Regis Sodoyer, Saint-Genis Les Ollieres (FR); Isabelle Peubez, Saint-Pierre La Palud (FR); Raphaelle El Habib, Chaponost (FR)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SANOFI PASTEUR, Lyon (FR)

(21) Appl. No.: 12/532,307

(22) PCT Filed: Mar. 17, 2008
(86) PCT No.: PCT/EP08/53168

§ 371 Date: Sep. 21, 2009

Related U.S. Application Data
(60) Provisional application No. 60/896,553, filed on Mar. 23, 2007.

(51) Int. Cl. A61K 39/00; C07K 14/00; C07H 21/00; C12N 15/74; C07K 16/08; A61K 31/7088; A61P 31/18; A61P 37/04

(52) U.S. Cl. 424/185.1; 530/324; 530/350; 536/23.1; 435/320.1; 530/388.1; 514/44 R

(57) ABSTRACT

The invention relates to a polypeptide recognized by anti HIV-1 gp41 antibodies isolated from Highly Exposed, non Infected Women, and epitopic fragments and conjugate thereof. Method of inducing antibodies neutralizing HIV-1 against HIV-1 is also contemplated.

The invention relates to a new polypeptide recognized by anti HIV-1 gp41 antibodies isolated from Highly Exposed, non Infected Women, and its use to induce antibodies neutralizing against HIV-1.

Several studies indicated a correlation between the presence of secreted IgA in vaginal secretions and resistance to infection. Mazzoli et al. (Nat Med. 1997; 3(11):1250-7) identified HIV-1 specific IgA, but no IgG, in vaginal samplings from seronegative partners of seropositive men. Infected partners have been shown to produce both IgG and IgA. Such observations were confirmed by Kaul et al. (AIDS. 1999; 13(1):23-9). In addition, Bomsel et al. (Immunity. 1998; 9(2):277-87) have shown the possibility to block transcytosis of HIV primary isolates through the epithelial barrier by IgM or dimeric IgA.

Thus the identification of polypeptide(s) recognized by anti-HIV antibodies produced at the mucosal surface, potentially capable of viral neutralization, offers the perspective to define an immunogen or a vaccinal formula inducing a specific anti-HIV-1 immune response.

IgG and IgA antibody responses to HIV-1, from a few highly exposed non-infected haïtian women, were analysed and cloned through molecular techniques. Surprisingly, the isolated antibodies (mainly IgAs) displayed, upon analysis by sequencing, an oligoclonal profile and were shown to bind to the HIV-1 gp41 protein. Recombinant Fab fragments showing a significant in vitro neutralizing activity have been produced in either E. coli or a cell-free translation system. The epitopes recognized by these Fabs have been mapped and a novel polypeptide was identified flanking the membrane spanning domain.

The present invention thus provides a polypeptide comprising SEQ ID No1 or a functional polypeptide variant thereof.

According to a particular embodiment, the polypeptide of the invention comprises SEQ ID No 5
Wherein:
Xa represents A, T, S, V, or I, and
Xb represents I, L, V or T
or a functional polypeptide variant thereof.

According to a specific embodiment, the invention provides a polypeptide comprising SEQ ID No 5 wherein Xa represents A and Xb represents I, or a functional polypeptide variant thereof.

According to another embodiment, the polypeptide of the invention comprises SEQ ID No 6
Wherein:
Xa represents A, T, S, V, or I,
Xb represents I, L, V or T, and
X1 represents I or V
X2 represents I or V
X3 represents L or I,
X4 represents V, I, A, or M,
X5 represents N or K,

X6 represents L, V or I,
X7 represents F or L, and
X8 represents T or I, or a functional polypeptide variant thereof.

According to a specific embodiment, the invention provides a polypeptide comprising SEQ ID No6 wherein Xa represents A, Xb represents, X1 represents I or V, X2 represents I or V, X3 represents L, X4 represents V, X5 represents N, X6 represents L, X7 represents F, and X8 represents T.

According to a particular embodiment, the polypeptide of the invention comprises SEQ ID No7:
Wherein:
Xa represents A, T, S, V, or I,
Xb represents I, L, V or T,
X9 represents I or V,
X10 represents G, I, or V,
X11 represents I, L or V,
X12 represents V, I, A, or M,
X13 represents N or K,
X14 represents L, V or I, and
X15 represents F or L, or functional variants thereof.

According to a specific embodiment, the invention provides a polypeptide comprising SEQ ID No7 wherein Xa represents A,Xb represents I, X9 represents I or V, X10 represents V, X11 represents I, X12 represents V, X13 represents N, X14 represents L, and X15 represents F.

According to another embodiment, the invention provides a polypeptide as defined above, further comprising an amino acid sequence SEQ ID No8 linked by an amide bond at its C-terminal end
Wherein:
X16 represents H, L, P, R or Q,
X17 represents L, T, F, I, S, P, A, C or N,
X18 represents P or H,
X19 represents T, A, N, H, I, S, Q, V or D,
X20 represents A or absent,
X21 represents P, Q, S, R, A or T,
X22 represents R, G, K, E or P,
X23 represents G, E, D, R or P, and
X24 represents P, L, F, H or T, or a functional polypeptide variant thereof.

According to a specific embodiment, the invention provides a polypeptide comprising SEQ ID N^{o}8 wherein X16 represents H, X17 represents L, X18 represents P, X19 represents T or A, X20 is absent, X21 represents P, X22 represents R, X23 represents G and X24 represents P.

According to another aspect, the present invention provides a conjugate comprising a polypeptide or a functional polypeptide variant thereof as defined above, conjugated to a carrier protein.

According to another aspect, the present invention provides a nucleic acid comprising a sequence encoding a polypeptide or a functional polypeptide variant as defined above or a conjugate as defined above.

According to another aspect the present invention provides an expression vector comprising a nucleic acid as defined above.

According to another aspect, the present invention provides an immunogenic composition comprising a polypeptide or a functional polypeptide variant thereof, a conjugate, a vector, as defined above, a pharmaceutically acceptable carrier, and optionally an adjuvant.

According to another aspect the present invention provides a method of inducing a specific immune response against HIV-1 in a mammal comprising administering said mammal with an immunogenic composition as defined above.

According to another aspect the present invention provides a monoclonal antibody characterized by a VL variable region sequence consisting of SEQ ID No 3 and a VH variable region sequence consisting of SEQ ID No 4 or any fragment thereof containing at least SEQ ID No 3 and SEQ ID No 4.

According to another aspect the present invention provides an immunogenic composition comprising a monoclonal antibody or a fragment thereof as defined above and a pharmaceutically acceptable carrier, and optionally an adjuvant.

According to another aspect, the present invention provides a method of inducing specific passive immunization against HIV-1 in a mammal comprising administering said mammal with an immunogenic composition comprising a monoclonal antibody or a fragment thereof as defined above.

The present invention is going to be described in more details in the description below.

### Definitions

According to a first aspect, the present invention thus provides a polypeptide comprising, or consisting of, the amino acids 181 to 208 of gp41 protein of HIV-1 isolate HXB2 (SEQ ID No1), as well as any functional polypeptide variants thereof.

Numerotation is based on the gp-41 protein of HIV-1 isolate HXB2 (SEQ ID No2) taking as number 1, Ala 1.

By polypeptide we mean here a chain of amino acids linked by amide bond comprising from 6 to 100, in particular from 6 to 50, such as 10 to 40, 15 to 40 and more particularly from 20 to 40 amino acids.

By gp41 protein we mean a gp41 glycoprotein from a HIV-1 strain, including laboratory strains as well as primary isolates of any circulating clades, such as clade A, B, C, D, E, in particular from clade B. Nucleotidic and peptidic sequences of a large number of gp41 proteins are known and available on the net. See for example the compendia from Los Alamos. An example of a gp41 sequence is shown as SEQ ID No.2 (gp-41 of HIV-1 isolate HXB2).

Alignment of the polypeptide sequence of the invention (SEQ ID No 1 with gp41 sequences of other HIV-1 strains from different clades showed that most of the amino acids contained in the polypeptide of the invention are highly conserved (i.e. for a given position variation are rarely observed and when observed correspond only to conservative substitution).

gp41 glycoprotein is usually 345-amino-acid-long. However, HIV-1 variant strains may also exist with missing or additional amino acid(s). Using the sequence SEQ ID No1 as defined in the present application, the person skilled in the art can thus easily identify polypeptide variants.

All the functional polypeptide variants of the polypeptide of the invention are within the scope of the invention.

By functional polypeptide variants we mean here all polypeptide variants which are specifically recognized by the Makandal monoclonal antibody (MMab) identified from highly exposed non infected haïtian women. The MMab is characterized by a VL variable region sequence consisting of SEQ ID No 3 and a VH variable region sequence consisting of SEQ ID No 4. The said monoclonal antibody can be easily produced by one person skilled in the art using the above SEQ ID No3 and 4 sequences and the standard monoclonal antibody construction/production methods.

A polypeptide variant is recognized specifically by the MMab when in a standard ELISA assay the said polypeptide variant binds to a Fab fragment to the said MMab thus leading to a positive result. Briefly To determine if a variant is a functional polypeptide variant according to the invention, an ELISA plate is coated with the Fab fragment of the MMab, non binding sites are then saturated with BSA or another blocking reagent, the biotinylated polypeptide variant is then incubated at least 1 H at 37° C. and after several washing steps, the thus—formed complexes are then detected by a standard colorimetric reaction using a densitometric measurement. All these steps are conducted using standard conditions well-known by the person skilled in the art.

According to an embodiment the present invention thus provides a polypeptide comprising, or consisting of, the sequence: SEQ ID No 5

LVGLRIVFXaVLSXbVNRVRQGYSPLSFQT

Wherein:
Xa represents A, T, S, V, or I, preferably A, and
Xb represents I, L, V or T, preferably I.

According to a preferred embodiment the polypeptide of the invention thus comprises, or consists of, the sequence SEQ ID No 5 wherein Xa represents A and Xb represents I.

According to another embodiment, the present invention thus provides a polypeptide comprising, or consisting of, the sequence: SEQ ID No 6

LX1GLRIX2FXaVX3SXbX4X5RVRQGYSPX6SX7QX8

Wherein:
Xa represents A, T, S, V, or I, preferably A
Xb represents I, L, V or T, preferably I, and
X1 represents I or V
X2 represents I or V
X3 represents L or I, preferably L,
X4 represents V, I, A, or M, preferably V,
X5 represents N or K, preferably N,
X6 represents L, V or I, preferably L,
X7 represents F or L, preferably F, and
X8 represents T or I, preferably T.

According to a preferred embodiment, the polypeptide of the invention thus comprises, or consists of, the sequence SEQ ID No 6 wherein Xa represents A, Xb represents, X1 represents I or V, X2 represents I or V, X3 represents L, X4 represents V, X5 represents N, X6 represents L, X7 represents F, and X8 represents T.

According to another embodiment, the present invention thus provides a polypeptide comprising, or consisting of, the sequence SEQ ID No7:

LX9GLRIVFXaX10LSXbX11X12X13RVRQGYSPX14SX15QT

Wherein:
Xa represents A, T, S, V, or I, preferably A
Xb represents I, L, V or T, preferably I,
X9 represents I or V,
X10 represents G, I, or V, preferably V,
X11 represents I, L or V, preferably I,
X12 represents V, I, A, or M, preferably V,
X13 represents N or K, preferably N,
X14 represents L, V or I, preferably L, and
X15 represents F or L, preferably F.

According to a preferred embodiment, the polypeptide of the invention thus comprises, or consists of, the sequence SEQ ID No 7 wherein Xa represents A,
Xb represents I, X9 represents I or V, X10 represents V, X11 represents I, X12 represents V, X13 represents N, X14 represents L, and X15 represents F.

According to another embodiment, the polypeptides of the invention comprising, or consisting of, the sequence SEQ ID No5, SEQ ID No6 or SEQ ID No7 as defined above, further comprise an amino acid sequence SEQ ID No8 linked by an amide bond at its C-terminal end.
The SEQ ID No 8 consists of X₁₆X₁₇X₁₈X₁₉X₂₀X₂₁X₂₂X₂₃X₂₄
Wherein:
X16 represents H, L, P, R or Q, preferably H,
X17 represents L, T, F, I, S, P, A, C or N, preferably L,
X18 represents P or H, preferably P,
X19 represents T, A, N, H, I, S, Q, V or D preferably, T or A,
X20 represents A or absent, preferably absent,
X21 represents P, Q, S, R, A or T, preferably P,
X22 represents R, G, K, E or P, preferably R,
X23 represents G, E, D, R or P, preferably G and
X24 represents P, L, F, H or T, preferably P.

According to a particular embodiment, the polypeptides of the invention comprising, or consisting of, the sequence SEQ ID No5, SEQ ID No6 or SEQ ID No7 as defined above, further comprise an amino acid sequence SEQ ID No8 linked by an amide bond at its C-terminal end wherein
X16 represents H, X17 represents L, X18 represents P, X19 represents T or A, X20 is absent, X21 represents P, X22 represents R, X23 represents G and X24 represents P.

All the functional polypeptide variants of the above defined polypeptides of the invention are encompassed within the scope of the present invention.

### Identification of Polypeptide(s) Recognized by Anti-HIV Antibodies Produced at the Mucosal Surface

Samples of mucosal B cells from a cohort of people in the Republic of Haiti (non-infected women with seropositive partners) were collected. They included negative (non exposed-non infected) and positive (exposed-infected) controls.

Following ELISA and Western Blot analyses, antibody library construction focused on two subjects. IgA, IgG1 and light chain libraries were obtained and a surprisingly “oligoclonal” profile was observed upon sequencing of randomly picked clones. At first, a limited number of clones (2 IgAs, 2 IgGs) were selected for further functional evaluation through Fab production and in vitro neutralization test.

Sequences encoding the Fab fragments of the “best”candidate antibodies were assembled into different E. coli expression vectors. The “best” candidates were defined as the samplings with the highest level of antibody response against gp41 by ELISA and Western blot.

The expressed Fab fragments were screened against random peptide libraries displayed at the surface of filamentous bacteriophages.

Upon screening (biopanning) two overlapping peptides were recognized and specifically bound by the Fab fragment of the MMab. They are included into the region located from amino acids 181 to 216 of HIV-1 gp41 and thus allowed us to define a new polypeptide of interest for inducing an immunization against HIV-1.

The polypeptides and functional polypeptide variants thereof according to the invention may be obtained by any conventional technique of chemical synthesis or of genetic engineering well known by the person skilled in the art.

When the polypeptides and functional polypeptide variants thereof can be produced by chemical synthesis, it may be synthesized in the form of a single sequence, or in the form of several sequences which are then linked to one another. The chemical synthesis may be carried out in solid phase or in solution, these two synthesis techniques being well known to those skilled in the art. These techniques are in particular described by Atherton and Shepard in “Solid phase peptide synthesis” (IRL press Oxford, 1989) and by Houbenweyl in “Methoden der organischen Chemie” [Methods in Organic Chemistry] published by E. Wunsch Vol. 15-I and II, Stuttgart, 1974, and also in the following articles, which are entirely incorporated herein by way of reference: P E Dawson et al. (Science 1994; 266(5186):776 9); G G Kochendoerfer et al. (1999; 3(6):665 71); et P E Dawson et al., Annu. Rev. Biochem. 2000; 69:923 60.

The polypeptides and the functional polypeptide variants thereof according to the invention may also be produced using genetic engineering techniques well known to those skilled in the art. When the said polypeptides according to the invention are produced by genetic engineering, it may comprise, at the NH2-terminal end, an additional methionine residue corresponding to the translation of the first initiation codon.

These techniques are described in detail in Molecular Cloning: a molecular manual, by Maniatis et al., Cold Spring Harbor, 1989. Conventionally, the PCR technique is used to produce the DNA sequence encoding the polypeptides and functional polypeptide variants thereof according to the invention in a form which can be inserted into an expression vector. The expression vector containing the sequence of interest is then used to transform a host cell which allows for expression of the sequence of interest. The polypeptides and the functional polypeptide variants thereof produced are then isolated from the culture medium using conventional chromatography techniques well known to those skilled in the art. High performance liquid chromatography (HPLC) is preferably used in the purification. Typically, the cells are collected by centrifugation at the end of culture, and are taken up in a neutral buffer, in order to be disrupted by any suitable means. The cell lysate is then centrifuged at approximately 10 000 g in order to separate the soluble material from the insoluble material. SDS-PAGE analysis of the supernatant and of the pellet from centrifugation will reveal whether the polypeptide is soluble or not. If the peptide or epitopic fragment is insoluble, solubilization is obtained using a buffer containing urea, guanidine or other solubilizing agents. Centrifugation at this step makes it possible to remove debris and other insoluble products which would hamper the chromatography.

The following step consists in loading the solubilized molecule onto an affinity column, which may be of the metal chelate type if a polyhistidine tail is integrated onto the polypeptide of interest. The system which enables the affinity purification may be varied in nature, such as immunoaffinity, affinity on cibachron blue, etc. At this stage, the protein exhibits a degree of purity close to or greater than 80%, as may be determined by SDS PAGE electrophoresis followed by Coomassie blue staining. An additional chromatography step may be added in order to further purify the polypeptide; by way of example, mention may be made of gel filtration and reverse-phase chromatography.

The polypeptides and the functional polypeptide variants according to the invention may thus be obtained in purified form, i.e. in a form exhibiting a degree of purity of at least 80%, in particular of at least 90%. The degree of purity is defined relative to the other proteins present in the mixture which are considered to be contaminants. This degree is evaluated by colorimetry of an SDS-PAGE using Coomassie blue. Densitometric measurement of the bands makes it possible to quantify the degree of purity. The degree of purity may also be measured by reverse-phase HPLC, by measuring the area of the various peaks.

According to a second aspect, the invention provides a conjugate comprising a polypeptides or functional polypeptide variants thereof as defined above, conjugated to a carrier protein.

The carrier protein strengthens the immunogenicity of the polypeptide according to the invention, in particular by increasing the production of specific antibodies. Said carrier protein preferably comprises one or more T helper epitope(s).

Examples of suitable carrier protein include bacteriophage surface proteins, such as the pIII or pVIII proteins of the M13 phage, coat protein of the bacteriophage MS2 or Q-beta, bacterial surface proteins, such as the LamB, OmpC, ompA, ompF and PhoE proteins of E. coli, the CotC or CotD protein of B. subtilis, bacterial porins, such as Neisseria gonorrheae porin P1, H. influenzae B porin P1 or P2, N. meningitidis B class I porin or K. pneumoniae porin P40, lipoproteins, such as B. bugdorfi OspA, S. pneumoniae PspA, N. meningitidis B TBP2, E. coli TraT and also S. pneumoniae adhesin A, and the heat shock proteins, such as Hsp65 or Hsp71 of M. tuberculosis or bovis, or Hin 47 of H. influenzae type B. Detoxified bacterial toxins, such as tetanus or diphtheria toxoid, the cholera toxin B subunit, or the B subunit of P. aeruginosa endotoxin A or S. aureus exotoxin A, are also suitable. Alternatively, the polypeptides and the functional polypeptide variants thereof could be linked to of the ectodomaine of a HIV-1 gp41 protein or to a bacterial surface protein, or it could be presented in a multimeric form. As a carrier peptide, use may be made, for example, of the p24E, p24N, p24H and p24M peptides described in WO 94/29339 and also the PADRE peptides as described by Del guercio et al. (Vaccine (1997); Vol. 15/4, p. 441 448).

The carrier protein (or peptide) is linked to the N- or C-terminal end of the polypeptides or functional polypeptide variants thereof according to the invention using any conjugation method well known to those skilled in the art. In addition, the sequence encoding the carrier protein (or peptide) may advantageously be fused to the sequence encoding the polypeptide and variants according to the invention, and the resulting sequence may be expressed in the form of a fusion protein using conventional process. All the genetic engineering techniques which can be used to do this are described in Maniatis et al. Said conjugates can be isolated using any conventional purification process which is well known to those skilled in the art.

According to a third aspect, the invention relates to an isolated nucleic acid (i.e. DNA) comprising or consisting of a sequence encoding a polypeptide, a functional polypeptide variant thereof or a conjugate as defined above. The DNA sequences according to the invention may be easily produced by PCR using, as a matrix, the nucleotide sequence of a HIV-1 gp41 protein.

A fourth aspect of the invention concerns an expression vector comprising a DNA encoding a polypeptide, a functional polypeptide variant thereof or a conjugate as defined above.

The terms “vector” and “expression vector” mean the vehicle by which a

DNA or RNA sequence (e.g. a foreign gene) can be introduced into a host cell, so as to transform the host and promote expression (e.g. transcription and translation) of the introduced sequence. Vectors include plasmids, phages, viroses, etc.

Use is preferably made of vectors in which the DNA sequence of the polypeptide or variant according to the invention is under the control of a strong promoter, which may or may not be inducible. By way of example of a promoter which may be used, mention may be made of the T7 RNA polymerase promoter.

The expression vectors may include a selectable marker, such as the kanamycin, tetracycline or other antibiotic resistance genes appropriate for use in humans. Alternatively the selection might be due to an auxotrophic marker, or any kind of antibiotic-free selection means (complementation of an essential gene previously knocked-out into the host's genome).

Examples of expression vector which may be used include the plasmid pET28 (Novagen) or pBAD (Invitrogen), yeast, bacteria, viral vectors, such as: baculoviruses, poxviruses, in particular the poxviruses described in patents U.S. Pat. No. 5,942,235, U.S. Pat. No. 5,756,103 and U.S. Pat. No. 5,990,091, and recombinant vaccinia viruses, in particular the recombinant viruses described in patents EP 83286, U.S. Pat. No. 5,494,807 and U.S. Pat. No. 5,762,938.

In order to promote the expression and purification of the polypeptide, the latter may be expressed in a modified form, such as a fusion protein, and may include not only secretion signals, but also additional heterologous functional regions. For example, a region of additional amino acids, particularly charged amino acids, may be added at the N-terminal of the polypeptide in order to improve stability and persistence in the host cell. The invention also provides a host cell transformed with said vector. Any host cell conventionally used in combination with the expression vectors described above may be used, for instance E. coli, BL21 (lamdaDE3), HB101, Top 10, CAG 1139, Bacillus or other gram positive hosts such as Lactococcus lactis, yeast, baculovirus and eukaryotic cells such as CHO or Vero. Preferred expression systems include pET/BL21 LamdaDE3 and BL21lamdaDE3(RIL).

According to a fifth aspect, the present invention provide a monoclonal antibody characterized by a VL variable region sequence consisting of SEQ ID No 3 and a VH variable region sequence consisting of SEQ ID No 4 as well as any fragment thereof containing at least SEQ ID No 3 and SEQ ID No 4.

Immunogenic Compositions

The invention further relates to an immunogenic composition comprising a polypeptide, a functional polypeptide variant thereof, a conjugate, a vector or a MMab or a fragment thereof as defined above, and a pharmaceutically acceptable carrier or diluent and optionally an adjuvant. Preferably, the immunogenic composition is a vaccine. Said immunogenic composition or vaccine may be used for prophylactic or therapeutic immunization against HIV-1 infection.

The immunogenic compositions or vaccines according to the present invention may be prepared using any conventional method known to those skilled in the art. Conventionally the polypeptide, the functional polypeptide variant thereof, the conjugate or the vector according to the invention is mixed with a pharmaceutically acceptable diluent or excipient, such as water or phosphate buffered saline solution. The excipient or diluent will be selected as a function of the pharmaceutical form chosen, of the method and route of administration and also of pharmaceutical practice. Suitable excipients or diluents and also the requirements in terms of pharmaceutical formulation, are described in Remington's Pharmaceutical Sciences, which represents a reference book in this field.

Preferably, the immunogenic composition or vaccine corresponds to a liquid composition comprising an aqueous buffered solution to maintain e.g. a pH (as determined at RT with a pH meter) in the range of 6 to 9.

The composition according to the invention may further comprise an adjuvant, i.e. a substance which improves, or enhances, the immune response elicited by the polypeptide, functional polypeptide variant thereof, or conjugate. A mucosal adjuvant may advantageously be used such as the LT (heat labile toxin) of E. coli or the CT (cholera toxin)-derivatives

The immunogenic compositions or vaccines according to the invention may be administered by any route used in the field of mammalian vaccines, such as the parenteral (e.g. intradermal, subcutaneous, intramuscular), or preferably the mucosal route in particular by nasal, vaginal or rectal route.

The amount of polypeptide, functional polypeptide variant thereof, conjugate, vector, or MMab in the composition according to the present invention depends on many parameters, as will be understood by those skilled in the art, such as the nature of the carrier protein, the vector used or the route of administration. A suitable amount is an amount such that a specific mucosal immune response is induced after administration of said composition. The amount of polypeptide, functional polypeptide variant thereof that may be administered is of the order of 10 μg to 5 mg, the amount selected varying depending on the route of administration. The amount of conjugate that may be administered will be deduced from the amounts indicated above, taking into account the MW of the carrier protein. The amount of expression vector that may be administered is of the order of 10 to 5000 μg in the case of a nonviral vector, and of the order of 10E4 to 10E8 TCID50 in the case of a viral vector and in the order of 1 to 10 000 mg for the MMab or fragment thereof.

### Methods of Inducing Antibodies Neutralizing HIV-1 or Conferring Mucosal Protection

The immunogenic compositions according to the invention may elicit a specific mucosal immune response toward HIV-1 virus (in particular primary isolates), comprising neutralizing antibodies, in particular specific IgA.

The induction of antibodies which neutralize HIV-1 virus can be determined using the neutralization test as described in the article by C. Moog et al. (AIDS Res. Hum. retroviruses, Vol. 13(1), 19-27, 1997). In the context of the present invention, it is estimated that neutralizing antibodies have been induced by the antigen tested according to the technique of C. Moog when the vaginal washes diluted at least to ¼ or recombinant Fab fragment diluted to at least ½, in the presence of HIV, leads to a 10-fold decrease in the viral titer in comparison to HIV alone, the viral titer being evaluated by the amount of p24 produced in the culture supernatant.

The induction of antibodies which neutralize HIV-1 virus may also be determined using the neutralization test of D. Montefiori (J. Infect. Dis. 1996, 173:60 67). In this test, the neutralizing titer is expressed by the percentage decrease in p24 antigen produced in the culture supernatants when the virus is incubated in the presence of vaginal washes diluted to ¼, by comparison with the virus in the absence of vaginal washes. In the context of the present invention, it is considered that neutralizing antibodies have been induced when the decrease in the level of p24 produced reaches at least 80% with a vaginal washes diluted to ¼.

It is considered that the antibodies induced by the polypeptide, functional polypeptide variant thereof or conjugate according to the invention are neutralizing antibodies if neutralizing activity is detected for a given HIV-1 virus in at least one of the two tests above.

Additionally, the polypeptide of the invention has been identified as associated with anti-HIV gp41 antibodies (mainly IgA) present in vaginal washes of highly exposed HIV-seronegative women. These antibodies are thought to be potentially responsible for mucosal protection against HIV-1 infection.

Therefore, the invention also relates to a method of inducing a mucosal immune response against HIV-1 in a mammal, comprising administering said mammal with an immunogenic composition as defined above.

A “mammal” is intended for a human or non human mammal.

The term mucosal immune response is intended to mean a response comprising at least production of IgA directed specifically against the polypeptide of the invention in at least one mucosal site, such as vaginal, rectal, preferably in vaginal secretions. The production of specific antibodies can be easily determined using conventional techniques which are well known to those skilled in the art, such as ELISA, IRA, or Western Blot.

The invention will be further illustrated in the Examples.

### EXAMPLES

A Clinical protocol was designed in 1998-1999 to organize the collect of samples of mucosal B cells from a cohort of people in the Republic of Haïti (non-infected women with seropositive partners). The follow up of the cohort was done by GHESKIO (Groupe Haïtien d'Etude du Sarcome de Kaposi et des Infections Opportunistes) center in Port au Prince. A total of 138 vaginal samples were collected and received. They included negative (non exposed-non infected) and positive (exposed-infected) controls as well as several re-samplings for some of the candidates according to their specific antibody response. A significant number of samples containing Prostate Specific Antigen were discarded to avoid collecting B cells of the partner.

### Example 1

### Analysis of the CCR5 co-Receptor Genotype

In the context of the study, it was important to show that the protection against HIV infection was not related to a defect on the gene encoding the CCR5 co-receptor, a co-receptor necessary for HIV entry into cells.

Accordingly, a CCR5 co-receptor analysis was performed on 48 samples obtained from a group of exposed non-infected women as well as negative controls.

Among the 48 samples analysed, 46 were w+/w+, 2 were w+/δ32 (1 out of the 23 controls, 1 out of the 25 sero-different) but the δ32/δ32 genotype was never encountered. The frequency of the δ32 allele was not different between the control group (2.17%) and the sero-different group (2.00%). Interestingly the CCR5 typing of the selected subjects was w+/w+.

### Example 2

### Sperm Dosage

Sperm contamination was tested in parallel in 2 different laboratories. For the vast majority of tested samples, a good correlation was observed between the results obtained from both laboratories. However, a few discrepancies occurred, for some samples. In order to avoid any doubt, samples positive in one test were systematically discarded.

### Example 3

### ELISA and Western Blot Analysis of IgAs and IgGs

For Western blot analyses, a Lay Blot I kit was used according to the manufacturer's instructions.

ELISA were performed as described in Alfsen et al. (The Journal of Immunology, 2001, 166: 6257-6265). Briefly, plates were coated overnight at 4° C. with 100 μl/well of either recombinant HIV-1 envelope glycoprotein gp160 (2.5 μg/ml) or HIV-1 peptides (0.2 μg/ml) diluted in PBS. After blocking the plates with 3% skimmed milk and washing, purified S-IgA or IgG were added and incubated for 1 h at 37° C. After washing, rabbit HRP-labeled Abs to human F(ab′)2 ( 1/2000) were added for 1 h at 37° C. HRP activity was detected with o-phenylenediamine (Sigma, St. Louis, Mo.) and read at 492 nm with a Titertek Multiskan spectrophotometer (Flow Laboratories, Glasgow, U.K.). The results were compared with a standard curve of a pool of whey from human milk containing 460 μg/ml of S-IgA. A large pool of 250 normal human sera containing 14 mg/ml of IgG was used as serum standard. The specific activities of S-IgA and of IgG against gp160 were expressed as arbitrary units per mg of Igs.

The ratio of S-IgA/IgG anti-gp160=S-IgA specific activity for gp160/IgG specific activity for gp160 represents the relative amount of Abs in the two Ig subclasses. For the peptide specific activity analysis, the results for each cervicovaginal secretion (#) were expressed by the ratio of S-IgA/IgG anti-peptide=Qi/ratio of S-IgA/IgG anti-gp160, where Qi=S-IgA specific activity for peptide i/IgG specific activity for peptide i is the ratio of the specific activity of S-IgA to that of IgG for the various HIV-1 envelope glycoprotein peptide i. Therefore, the ratio of S-IgA/IgG anti-peptide is Qi normalized to the total amount of anti-HIV-1 envelope glycoprotein Abs contained in S-IgA subclass relative to IgG.

Based on these analyses, two candidates were selected. Subject #74 was considered as the best candidate because very strong ELISA responses (either IgA or IgG) and strong responses in Western Blotting were found. Furthermore the subject had had long term relationships with a seropositive partner including her husband who deceased in 2000, yet the subject was still seronegative several months after (May 2001). The CCR5 co-receptor genotype was found w+/w+. Subject #39 was another interesting candidate considering the ELISA titer (especially IgA) and also according to it's markedly oligoclonal profile of heavy chains.

### Example 4

### Antibody Cloning

From the 2 identified candidates (subjects #74 and #39), 4 antibody clones were selected for the assembly of constructs allowing for expression in E. coli or cell free translation. The list of clones is the following:

#74 IgG1 (Toussaint) in association with 2 different light chains

#74 IgA (Makandal) in association with 2 different light chains

#39 IgG1 (Jacmel)

#39 IgA (Mangue)

Construction of a Library of Sequences Encoding Variable Light or Heavy Chain

Vaginal samples were collected using a “cytobrush” device and immediately dropped into 1 ml of lysis buffer from μMacs mRNA Isolation kit (Miltenyi biotec)

Poly A+ mRNA preparation was performed using the pMacs mRNA Isolation Kit (Miltenyi Biotec). Single stranded cDNA was obtained using a cDNA cycle kit (Invitrogen) after annealing oligo DT.

cDNAs were PCR amplified using a two-step semi-nested PCR.

The first PCR used primers for specific amplification of heavy and light chain variable regions which had been previously described (Sodoyer et al., R, Hum Antibodies. 1997; 8 (1):37-42).

Downstream primers complementary to human J region were used for the IgG, IgA1 and IgA2 heavy chains amplification:

The amplification conditions were as follow: 95° C. 15 sec, 97° C. 30 sec, 55° C. 1 min, 72° C. 1 min for 45 cycles using the “hot start taq” from Qiagen.

In the second semi-nested PCR, 2 μl of PCR1 was used as a template. The primers were selected to allow specific amplification of heavy and light chain constant regions. The amplification conditions were the same as for the first PCR.

The PCR amplified VH fragments were digested with XhoI and Spe I, and VL fragments with XbaI and SacI, and cloned into pVH, a phagemid vector allowing for the cloning of both VH and VL sequences and bacteriophage surface display of antibody Fab fragments

Sequencing of the library was carried out on an automated sequencer (LICOR 4200) using SequinTherm excel DNA sequencing Kit (Epicentre) and fluorescent primers.

This analysis led to the identification of the Makandal monoclonal antibody (MMab) characterized by a VL variable region sequence consisting of the amino acid sequence SEQ ID No 3

ELTQPSSLSASPGASASLTCTLRSGINVGTYRIYWYQQKPGSPPQFLLRY
KSDSDKEQGSGVPRRFSGSKDASANAGILLISGLQSEDEADYYCMIWHSG
AWVFGGGTKLTVLS

and a VH variable region sequence consisting of the amino acid sequence SEQ ID No 4

LESGGGMVQPGRSLRLSCAVSGFTFDDYAMHWIRQVPGKGLEWVAGISWN
SVKVDYADAVRGRFTISRDNAKNSLHLEMSNLRRDDTAFYFCAKDGGPGM
TIGGYVVTGRFDPWGQGTLVHVSS.

Two expression systems have been used: E. coli was used for producing Fabs for the in vitro neutralization assay, and an acellular system was used for producing Fabs for epitope identification.

E. coli Based Expression System

A tricistronic vector was constructed for the expression of recombinant Fabs in E. coli

A proprietary vector derived from pET28 vector was modified to insert a cassette containing the following elements: Leader sequence OmpT-Xho Spe I cloning sites-Flag tag-RBS—leader sequence PeIB-SacI-XbaI cloning sites—a 6× His tag-RBS Fkpa encoding sequence (vector pSP502).

The VL chain was cloned between XhoI and Spe I, and the VH chain between Sac I and XbaI.

Cell-Free Translation

Selected heavy and light chains were cloned into vectors suitable for in vitro translation in a commercial kit allowing for disulfide bonds assembly and expression of correctly folded and assembled Fab fragments.

More specifically, the pIVEX2.4b NheI vector (Roche diagnostic) was modified to facilitate VH and VL cloning. XhoI, SacI, SpeI, and XbaI sites were inserted in the poly linker. pIVEX+VH and pIVEX+VL were created by cloning respectively the VH chain and the VL chain.

Rapid Translation System RTS 500 E. coli Disulfide Kit (Roche) was used to produce recombinant Fab by co-translation of the genes previously cloned into vectors pIVex+VH and pIVEX+VL, allowing for microgram amount production and disulfide bonds assembly.

Coupled transcription/translation reactions were conducted in vessels with 2 compartments (reaction compartment and feeding compartment) separated by semi-permeable membranes.

Into the feeding compartment were mixed:

lysate from E. coli containing components necessary for transcription/translation (T7 RNA polymérase, ribosomes . . . )

chaperonnes proteins GroEL et GroES, increasing the yield of correctly folded soluble proteins,

disulfide isomerase, allowing assembly and rearrangement of disulfide bonds,

a redox buffer, maintaining oxydative conditions to facilitate disulfide bond formation.

In vitro translation was used to produce also VH and VL alone. In addition a negative control of the reaction devoid of any DNA was done and used for the analysis of Fab produced and also preincubated with the peptide libraries in order to reduce background.

The Fab fragment produced by the Roche cell-free translation system was adsorbed on microtitration plates and incubated with the phage displayed combinatorial peptide library.

After 4 or 5 rounds of selection, individual clones retained for specific binding to the Fab fragment were analyzed by sequencing. The corresponding translated amino acid sequences were analyzed for matching with the gp41 primary protein sequence.

### Example 5

### In vitro Neutralization

Fab fragments, were prepared and submitted to the first round of HIV neutralization assay.

The neutralization test performed on PBMC (pool of five donors) was described in detail in (Moog et al., AIDS Res Hum Retroviruses. 1997 Jan. 1; 13(1):19-27).

The characteristics of this assay are that it combines serial dilutions of virus with serial dilutions of sera, instead of using a single defined viral input, and is based on detecting a 10-fold virus titer reduction in the presence of immune sera or IgG. The main advantages are that the influence of the amount of virus used in the assay on the antibody titer, as well as the effect of differences in the efficacy of virus replication in PBMC from different donors, is minimized.

Briefly, 50-μl aliquots of four fourfold dilutions of virus, beginning with about 800 50% tissue culture infectious doses (TCID₅₀)/ml, were incubated for 1 h at 37° C. with 50 μl of serial serum or IgG dilutions in a 96-well filtration plate (Durapor-Dv, 1.25-μm pore size; Millipore, Molsheim, France), before addition of 10⁵ PHA-stimulated PBMC (pool of five seronegative donors). After 2 h at 37° C., extensive washings (thrice with 200 μl of RPMI 1640) were done to remove unbound virus and antibodies. Cells were then cultured in 200 μl of RPMI 1640 containing 10% fetal calf serum and 20 IU of interleukin-2 per ml. Half of the medium was changed at 4 days post-infection, and viral production was measured at 7 days postinfection or sometimes later if the viral replication was weak. For each serum dilution, the assay was performed in quadruplicate and HIV-positive wells were identified by the level of p24 antigens (ELISA kit; Du-Pont or Innogenetics, Zwijndrecht, Belgium) in the culture supernatants. This allowed the viral titer (TCID₅₀) to be calculated, in the presence (Vₙ) and in the absence (Vₒ), of the serum, by the Reed and Muench method. Fifty percent neutralization corresponds to inhibition of virus replication in two wells which is insufficient to obtain a reproducible determination of the titer (Moog et al., AIDS Res Hum Retroviruses. 1997 Jan. 1; 13(1):19-27). Thus, the neutralization titer of the serum was defined as the reciprocal of the serum dilution that resulted in a 10-fold decrease in the viral titer (Vₙ/Vₒ=0.1). For a given dilution of serum, a neutralization percentage was also defined as 100−(Vₙ/Vₒ×100).

The sample concentration was evaluated using both Western Blotting with an anti-Fab monoclonal antibody and an anti gp41 ELISA assay.

TABLE 1
Tested samples

| | | Clone name - used E coli strain - final |
|---|---|---|
| n ^{o} | volume | concentration |
| 1 D | 1 ml | Positive control origami 3.5 μg/ml |
| 2 D | 1 ml | WN1 origami 2.3 μg/ml |
| 3 D | 1 ml | Toussaint VL I origami 138 ng/ml |
| 4 D | 1 ml | Toussaint VL D BL21 59 ng/ml |
| 5 D | 1 ml | Makandal VL I TG1 1 - 2 ng/ml |
| 6 D | 1 ml | Makandal VL D Rosetta Gami 60 ng/ml |
| 7 D | 1 ml | Jacmel Rosetta Gami 464 ng/ml |
| 8 D | 1 ml | Mangue BL21 528 ng/ml |
| 9 D | 1 ml | T20 0.97 mg/ml |

Sample 1 D was a positive control
Sample 2 D was a negative control

A standard in vitro, virus neutralization test was performed using a unique primary B isolate (Bx 08).

TABLE 2
In vitro neutralization assay

Unique cycle
(virus Bx08)

| | Sample Volume | Neutralisation | Toxicity |
|---|---|---|---|
| 1-D | 1 ml | <2 | <2 (x4) |
| 2-D | 1 ml | <2 | <2 (x4) |
| 4-D | 1 ml | <2 | <2 (x4) |
| 5-D | 1 ml | =2 | <2 (x4) |
| 6-D | 1 ml | <2 | <2 (x4) |
| 7-D | 1 ml | <2 | <2 (x4) |
| 8-D | 1 ml | 2 | =2 |
| 9-D | 1 ml | <2 | <2 (x4) |

Neutralisation < 2: no 90% decrease in the number of infected cells for a ½ dilution.
Neutralisation = 2: 90% decrease in the number of infected cells.
Toxicity < 2 (x4): 4 fold increase in cell death.
Toxicity = 2: 10 fold increase in cell death.

The results were interesting when considering the following:

The Fab fragments were expected to be less efficient, in neutralisation, than the corresponding full size antibodies. This being particularly true for dimeric IgAs for which we expect an important avidity effect.

All samples, including the controls, were at very low concentrations.

The only sample rising over the back-ground was the Makendal IgA clone derived from subject #74.

At comparable very low concentration and even slightly higher concentration, the positive control failed to show any neutralisation effect.

### Example 6

### Epitope Mapping

Epitope mapping was done using recombinant Fab fragments screened against a commercially available phage displayed peptide library, the Ph D-7 (or Ph D-12) phage Display peptide Library Kit (New England Biolabs).

Fabs obtained by cell-free translation were half-diluted in 0.05M carbonate-bicarbonate buffer and coated in Nunc Immunoplate Maxisorp.

For each round of panning, 2.10¹¹ cfu/ml were incubated 1 h at room temperature. After washing with PBS-tween elution was performed by addition of 0.2M glycine-HCl (pH=2.2)-1 mg/ml BSA. The eluted fraction was then neutralized with 1M Tris-HCl (pH=9).

The phages were amplified by addition of an E Coli ER 2537 preculture supplied in Ph D-7 (or Ph D-12) phage Display peptide Library Kit (New England Biolabs) and incubated 4 h at 37° C. with shaking.

Five rounds of panning were carried out before clone analysis.

The two overlapping isolated peptides have been aligned with the gp41 membrane proximal sequence. They are included into the region located from amino acids 181 to 216.

Sequence alignment was performed with 131 gp41 sequences from clades A to E HIV-1 viruses of phenotype R5 and X4 as well as with 43 gp41 sequences from clades B HIV-1 viruses of phenotype R5 and X4.

This allowed us to define a new polypeptide of interest.

TABLE 3
HIV-1 gp41 peptide consensus (131 gp41 sequences from clades A to E HIV-1 viruses of phenotype R5 and X4)

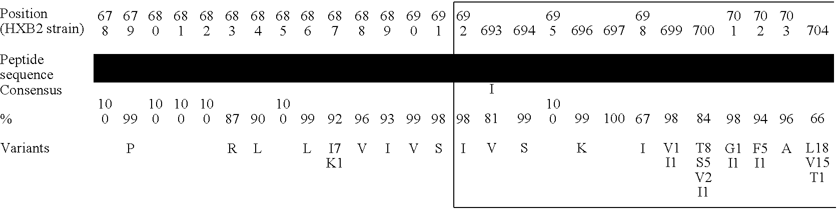

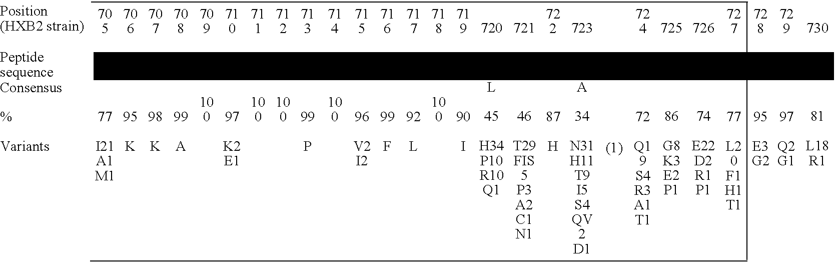

Boxed: peptide 692-727 on HXB2 genome

TABLE 4
HIV-1 gp41 peptide consensus (43 gp41 sequences from clades B HIV-1 viruses of phenotype R5 and X4)

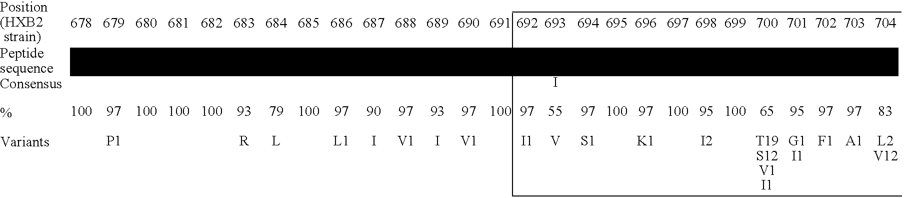

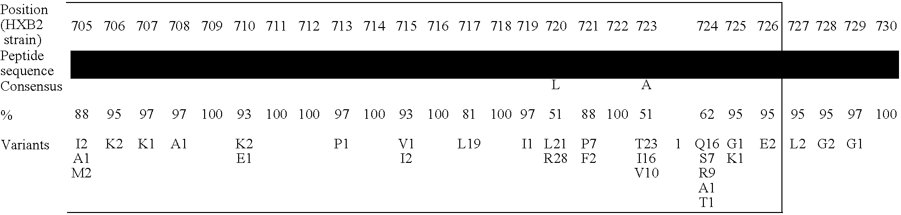

Boxed: peptide 692-727 on HXB2 genome

1. A polypeptide comprising SEQ ID No1 or a functional polypeptide variant thereof.

2. A polypeptide according to claim 1 comprising the sequence: SEQ ID No 5
LVGLRIVFXaVLSXbVNRVRQGYSPLSFQT
wherein:
Xa represents A, T, S, V, or I, and
Xb represents I, L, V or T
or a functional polypeptide variant thereof.

3. A polypeptide according to claim 2 wherein Xa represents A and Xb represents I.

4. A polypeptide according to claim 1 comprising the sequence: SEQ ID No 6
Wherein:
Xa represents A, T, S, V, or I,
Xb represents I, L, V or T, and
X1 represents I or V
X2 represents I or V
X3 represents L or I,
X4 represents V, I, A, or M,
X5 represents N or K,
X6 represents L, V or I,
X7 represents F or L, and
X8 represents T or I, or a functional polypeptide variants thereof.

5. A polypeptide according to claim 4 wherein Xa represents A, Xb represents, X1 represents I or V, X2 represents I or V, X3 represents L, X4 represents V, X5 represents N, X6 represents L, X7 represents F, and X8 represents T.

6. A polypeptide according to claim 1 comprising the sequence SEQ ID No7:
Wherein:
Xa represents A, T, S, V, or I,
Xb represents I, L, V or T,
X9 represents I or V,
X10 represents G, I, or V,
X11 represents I, L or V,
X12 represents V, I, A, or M,
X13 represents N or K,
X14 represents L, V or I, and
X15 represents F or L, or a functional polypeptide variant thereof.

7. A polypeptide according to claim 6 wherein Xa represents A, Xb represents I, X9 represents I or V, X10 represents V, X11 represents I, X12 represents V, X13 represents N, X14 represents L, and X15 represents F.

8. A polypeptide according to claim 1 further comprising an amino acid sequence SEQ ID No 8 linked by an amide bond at its C-terminal end.
Wherein:
X16 represents H, L, P, R or Q,
X17 represents L, T, F, I, S, P, A, C or N,
X18 represents P or H,
X19 represents T, A, N, H, I, S, Q, V or D,
X20 represents A or absent,
X21 represents P, Q, S, R, A or T,
X22 represents R, G, K, E or P,
X23 represents G, E, D, R or P, and
X24 represents P, L, F, H or T, or a functional polypeptide variant thereof.

9. A polypeptide according to claim 8 wherein X16 represents H, X17 represents L, X18 represents P, X19 represents T or A, X20 is absent, X21 represents P, X22 represents R, X23 represents G and X24 represents P.

10. A conjugate comprising a polypeptide or a functional polypeptide variant thereof according to claim 1, conjugated to a carrier protein.

11. A nucleic acid comprising a sequence encoding a polypeptide or a functional polypeptide variant thereof as defined claim 1.

12. An expression vector comprising a nucleic acid according to claim 11.

13. An immunogenic composition comprising a polypeptide or a functional polypeptide variant thereof as defined in claim 1, a pharmaceutically acceptable carrier, and optionally an adjuvant.

14. A method of inducing specific immune response against HIV-1 in a mammal comprising administering said mammal with an immunogenic composition according to claim 13.

15. A monoclonal antibody characterized by a VL variable region sequence consisting of SEQ ID No 3 and a VH variable region sequence consisting of SEQ ID No 4, any fragment thereof containing at least SEQ ID No 3 and SEQ ID No 4.

16. An immunogenic composition comprising a monoclonal antibody or any fragment thereof as defined in claim 15 and a pharmaceutically acceptable carrier, and optionally an adjuvant.

17. A nucleic acid comprising a sequence encoding a conjugate as defined in claim 10.

18. An immunogenic composition comprising a conjugate as defined in claim 10, a pharmaceutically acceptable carrier, and optionally an adjuvant.

19. An immunogenic composition comprising a a vector as defined in claim 12, a pharmaceutically acceptable carrier, and optionally an adjuvant.

20. A method of inducing specific immune response against HIV-1 in a mammal comprising administering said mammal with an immunogenic composition according to claim 18.

21. A method of inducing specific immune response against HIV-1 in a mammal comprising administering said mammal with an immunogenic composition according to claim 19.
